(19) United States
(12) Patent Application Publication
(10) Pub. No.: US 2025/0072721 A1
(43) Pub. Date: Mar. 6, 2025

(54) METHOD OF ASSEMBLING ARTICULATION SECTIONS OF A FLEXIBLE ENDOSCOPE AND DEVICE FOR PRACTICING THE METHOD

(71) Applicant: KARL STORZ SE & Co. KG, Tuttlingen (DE)

(72) Inventors: Robert TREMBLAY, Webster, MA (US); John MARCHESSAULT, Southbridge, MA (US)

(21) Appl. No.: 18/817,624

(22) Filed: Aug. 28, 2024

Related U.S. Application Data
(60) Provisional application No. 63/535,712, filed on Aug. 31, 2023.

(51) Int. Cl. A61B 1/00; A61B 1/005

(52) U.S. Cl. CPC A61B 1/0011; A61B 1/005

(57) ABSTRACT

A method includes providing a first articulation section with a connection edge with an inner rivet receiving hole and an opposite edge and a second articulation section having a connection edge with an outer rivet receiving hole and an opposite edge. The first articulation section is placed on a cam assembly loading pin with a cam surface. A rivet shaft of a rivet is placed into the inner rivet receiving hole with the head of the rivet facing the cam surface. The second articulation section is placed adjacent to the first articulation section with the outer rivet receiving hole aligned with the inner rivet receiving hole. The loading pin is rotated to press the cam surface against the rivet head to move the rivet shaft through the outer rivet receiving hole. The rivet is fixed to the second articulation section. A device is disclosed to practice the method.

### CROSS REFERENCE TO RELATED APPLICATIONS

This application claims the benefit of priority under 35 U.S.C. '120 of U.S. provisional patent application 63/535,712, filed Aug. 31, 2023, the entire contents of which are incorporated herein by reference.

### TECHNICAL FIELD

The invention relates to endoscopes and, more particularly, to a flexible endoscope and to a method of joining articulation sections of a flexible endoscope and relates to a device for practicing the method.

### TECHNICAL BACKGROUND

The conventional articulation section systems for an endoscope shaft employ an assembly method centered around the use of rivets to create a flexible and maneuverable structure. Such articulation section systems include several articulation sections interconnected through rivets, with each rivet featuring two heads. During assembly, the rivets are inserted into corresponding holes within the joining rings or ears from the inner side of the articulation sections, and subsequently, a second head is formed from the outer side.

A primary issue of such articulation section systems relates to the clearance between the rivet body and the two joining ring holes or ears, which adversely affects its torque resistance. Additionally, maintaining precise control over the dimensions of the rivet's cylindrical body during assembly may be difficult, leading to uncertainties in the clearance between the rivet head and the articulation sections.

One approach has been to weld the cylindrical body or rivet shaft of each rivet to the inner flat surface of one of the articulation sections, resulting in a single head located outside the articulation sections frame. However, the welding process presents difficulties as to maintaining the desired clearance due to the force required to press the rivet onto the ring's flat surface, which can inadvertently deform the ring and cause clearance issues.

U.S. Pat. Nos. 8,579,801 (B2) and 8,834,356 (B2) are said to present a method that can ensure better and consistent guaranteed clearance between the rivets and the section rings, thereby enhancing the overall performance and reliability of conventional endoscope shaft articulation sections. The method involves aligning fastener holes of two endoscope articulation section frame members with each other on a mandrel. The mandrel has a front end and a rear end. A fastener (rivet) is inserted into the fastener holes, wherein the fastener holes are aligned by moving the two endoscope articulation section frame members on the mandrel from the front end. A portion of the fastener is pushed by a linear cam feature on the mandrel to move a body of the fastener in the fastener holes of the endoscope deflection section frame members based on a linear movement of the two endoscope articulation section frame members with each other on the mandrel. The fastener is welded from an outside direction of the frame members, to an exterior side of one of the endoscope articulation section frame members to thereby pivotably attach the endoscope deflection section frame members to each other.

The linear cam method has the disadvantage that the assembler must move the articulation section frame members to be joined along with the rivets along the assembly mandrel. This requires a great deal of moving components that all need to be aligned while in motion.

### SUMMARY OF THE INVENTION

It is an object of the invention to provide a method and device for loading of the articulation hinge point (rivet) into the articulation section of a flexible endoscope that avoids problems presented by prior art methods.

It is an object of the invention to provide a method and device for loading of the articulation hinge point (rivet) into the articulation section of a flexible endoscope that avoids a need to align numerous moving components during assembly.

According to the invention, a method is provided that comprises providing a first articulation section (also referred to as a next to be connected articulation section) comprising a first section connection edge with an inner ear comprising a recessed rivet receiving portion having an inner rivet receiving hole and having an opposite section edge. A second articulation section (also referred to as the further adjacent articulation section) is provided that comprises a second section connection edge with an outer ear comprising an outer rivet receiving portion having an outer rivet receiving hole and having an opposite section edge. The first articulation section is placed on a cam assembly tooling mandrel and loading pin, the loading pin having a cam surface configured to contact with a head of a rivet. A rivet shaft of a rivet is inserted into the inner rivet receiving hole of the inner ear, wherein the head of the rivet is located at an interior side of the inner ear and facing the cam surface. The second articulation section is disposed adjacent to the first articulation section with the outer rivet receiving hole of the outer ear aligned with the inner rivet receiving hole of the inner ear. The loading pin is rotated relative to the first and second articulation sections (such as with the first and second articulation sections stationary), to press the cam surface of the loading pin against the head of the rivet to move the rivet shaft partially through the inner rivet receiving hole of the inner ear and into and partially through the outer rivet receiving hole of the outer ear. The rivet is subsequently fixedly connected to the second articulation section.

The rivet is advantageously fixedly connected to the second (further adjacent) articulation section by welding the rivet, particularly the rivet shaft, to the outer ear of the second articulation section.

The first section connection edge of the first (next to be connected) articulation section further comprises another inner ear comprising another recessed rivet receiving portion having another inner rivet receiving hole. The second section connection edge of the second (further adjacent) articulation section further comprises another outer ear comprising another outer rivet receiving portion having another outer rivet receiving hole. Another rivet is provided.

Another rivet shaft of the other rivet is inserted into the other inner rivet receiving hole of the other inner ear. Another head of the other rivet is located at an interior side of the inner ear and facing the cam surface, wherein the cam surface is configured to contact with the head of the rivet and also to contact a head of the other rivet. The rotation of the loading pin simultaneously presses the cam surface of the loading pin against both the head of the rivet to move the rivet shaft into and partially through the outer rivet receiving hole of the outer ear and also the head of the other rivet to move the other rivet shaft into and partially through the other outer rivet receiving hole of the other outer ear.

A method of forming fixedly connected articulation sections of an endoscope that has a shaft including a flexible portion comprised of the fixedly connected articulation sections, may comprise:
providing a to be connected articulation section having a section distal or proximal edge with a first inner ear comprising a recessed rivet receiving portion having an inner rivet receiving hole and a second inner ear comprising a recessed rivet receiving portion having an inner rivet receiving hole and with a proximal or distal edge with a first outer ear comprising an outer rivet receiving portion having an outer rivet receiving hole and second outer ear comprising an outer rivet receiving portion having an outer rivet receiving hole;
providing a further articulation section adjacent to the to be connected articulation section, the further articulation section having a distal or proximal edge with a first inner ear comprising a recessed rivet receiving portion having an inner rivet receiving hole and a second inner ear comprising a recessed rivet receiving portion having an inner rivet receiving hole and with a proximal or distal edge with a first outer ear comprising an outer rivet receiving portion having an outer rivet receiving hole and a second outer ear comprising an outer rivet receiving portion having an outer rivet receiving hole;
placing the to be connected articulation section on a cam assembly tooling mandrel and loading pin, the loading pin having a cam surface configured to contact with a head of a first rivet and a head of a second rivet;
inserting a first rivet shaft of the first rivet into the inner rivet receiving hole of the first inner ear, wherein the first head of the first rivet is located at an interior side of the first inner ear and facing the cam surface;
inserting a second rivet shaft of the second rivet into the inner rivet receiving hole of the second inner ear, wherein the second head of the second rivet is located at an interior side of the second inner ear and facing the cam surface;
placing the further articulation section adjacent to the to be connected articulation section with the inner rivet receiving hole of the first inner ear aligned with the outer rivet receiving hole of the first outer ear and with the inner rivet receiving hole of the second inner ear aligned with the outer rivet receiving hole of the second outer ear;
rotating the loading pin to press the cam surface of the loading pin against the first head of the first rivet to move the first rivet shaft into and partially through the outer rivet receiving hole of the first outer ear and to simultaneously press the cam surface of the loading pin against the second head of the second rivet to move the second rivet shaft into and partially through the outer rivet receiving hole of the second outer ear,
fixedly connecting the first rivet to the further articulation section; and
fixedly connecting the second rivet to the further articulation section.

Fixedly connecting the first rivet to the further articulation section comprises welding the first rivet to the first outer ear of the further articulation section; and fixedly connecting the second rivet to the further articulation section comprises welding the second rivet to the second outer ear of the further articulation section.

In accordance with this disclosure, a method and device are disclosed, which aligns rivet holes in two endoscope articulation sections with each other. A rivet is inserted into these rivet holes, with one end (head) of the rivet positioned on the interior side of the first articulation section and the opposite end extending to the exterior side of the second articulation section. Instead of using a mandrel with a linear cam, a rotating cam is inserted into the articulation sections to align them and to contact the rivet head, thereby aligning the rivet relative to the articulation sections while maintaining the necessary clearance between the rivet head and the inner ring ear. Finally, the rivet is securely fixed to the second articulation section.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a perspective view of a flexible endoscope assembled with the method of assembling articulation sections according to this disclosure;

FIG. 2 is a perspective view showing several joined articulation sections;

FIG. 3 is a cross sectional view showing the joined articulation sections of FIG. 2;

FIG. 4 is a perspective view of a to be connected articulation section (first articulation section) with two hinge points, in the form of upper and lower rivet receiving holes each defined by an inner ear (recess rivet receiving ring portion), and showing the articulation section on a cam assembly tooling mandrel and loading pin;

FIG. 5 is a perspective view of the arrangement shown in FIG. 4, showing the insertion of a first rivet;

FIG. 6 is a perspective view with an arrow that indicates a rotation of the arrangement (cam assembly tooling mandrel and loading pin and articulation section) shown in FIG. 5;

FIG. 7 is a perspective view of the arrangement shown in FIG. 5 after the rotation and showing an insertion of a second rivet at an opposite side of the arrangement of the articulation section on the cam assembly tooling mandrel and loading pin;

FIG. 8 is a perspective view showing the positioning of further (other) articulation sections at a location adjacent to the first articulation section with the adjacent further articulation section (second articulation section) shown as transparent (dashed line) to show a recessed rivet receiving hole of a recessed rivet receiving ring portion aligned with an outer rivet receiving hole of an outer rivet receiving ring portion of the adjacent further articulation section;

FIG. 9 is a perspective view showing the positioned further articulation sections on a carrying support mandrel to position the further articulation sections at a location adjacent to the first articulation section as shown in FIG. 8;

FIG. 10 is a cross sectional view showing the positioned further articulation sections on a carrying support mandrel at a location adjacent to the first articulation section and with the first articulation section carried by a mandrel of the cam assembly tooling mandrel and loading pin just before rotation of the loading pin and also schematically showing a welding device that may be included with a device for practicing the method;

FIG. 11 is a perspective view of the device for practicing the method, with an arrow indicating the rotation of the loading pin with a tooling support to effect a pressing of the rivet head to move the rivet shafts (rivet cylindrical portions) into and through the outer rivet receiving hole at each side;

FIG. 12A is a cross-sectional view showing the cam assembly tooling mandrel and loading pin in a first state prior to pressing the rivets; and

FIG. 12B is a cross-sectional view showing the cam assembly tooling mandrel and loading pin in a second state, rotated 90° relative to the first state, showing the rivet shafts pressed through each outer rivet receiving hole.

### DESCRIPTION OF THE PREFERRED EMBODIMENTS

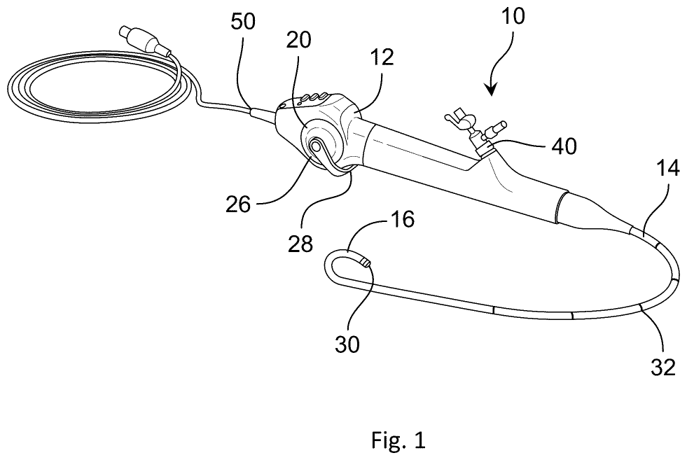

Referring to the drawings in particular, FIG. 1 shows a perspective view of an endoscope 10 with a flexible portion assembled with the method of assembling articulation sections according to this disclosure. The endoscope 10 may be a ureteroscope or other suitable type of endoscope that includes articulated sections. The endoscope 10 generally comprises a handle or control 12 and a deflection section 16 that is located at a distal end of the handle 12. The endoscope 10 has a shaft 14 with the deflection section 16. A control system 20 to control the active deflection section 16 extends from the handle 12 to the deflection section 16. The control system 20 can comprise, for example, a pair of control wires, two wire sheaths, and an actuator 26. One end of the wires are connected to the actuator 26 and an opposite second end of the wires are connected to the distal end of the active deflection section 16.

In the preferred embodiment, the handle 12 has a user operated slide or lever 28. The lever 28 is connected to the actuator 26. The actuator 26 is adapted to pull and release the two wires of the control system 20. When the lever 28 is moved by the user, the actuator 26 is moved. The actuator 26 may be a drum or pulley, for example, rotatably connected to the handle 12 to pull one wire while releasing the other. The actuator may also be a rocker arm adapted to pull and release the wires of the control system 20. The control system 20 may also have two or more pairs of control wires. In this implementation, the handle can have additional actuators and corresponding controls to drive the additional pairs of control wires to bend the deflection section in different plane(s). In further alternate embodiments, the handle 12 may have knobs with rack and pinion mechanisms or other suitable user operated controls for the control system.

The flexible shaft 14 with deflection section 16 includes the control wires of the control system 20, a fiber optical image bundle or video sensor electrical cable, a working channel, and a fiber optical illumination bundle or electrical wires to illumination LEDs or lights at an objective head 30. A port 40 for inserting accessory instruments (not shown) into the working channel is located on the handle 12. The handle 12 also has an electrical cable 50 for connection to another device, such as a video monitor for example. In an alternate embodiment, instead of the cable 50, the endoscope 10 could have an eyepiece. In alternate embodiments, the flexible shaft may house different systems therein.

The deflection section or steering/articulation section 16 generally comprises a frame that has a cover 32 and the objective head 30. The cover 32 also extends over the entire shaft 14, or over a portion or there may be different sections that have different cover portions. The frame has at least one portion comprising articulation sections 60 that are disposed adjacent to each other and are articulated to each other. The articulation sections 60 are comprised of stainless steel. The articulation sections 60 could be comprised of another material such as plastic or a shape memory alloy material, such as Tinel or Nitinol.

The articulation sections 60 each comprise a one-piece member, but could be comprised of multiple members. Each articulation section 60 has a first side connection edge 62, such as a proximal side connection edge, and a second side connection edges, such as a distal side connection edge 64. The first side connection edge 62 has an inner ear (recessed rivet receiving portion) 66 that defines an inner rivet receiving hole 68. The second side connection edge 64 has an outer ear (outer rivet receiving portion) 70 that defines an outer rivet receiving hole 72.

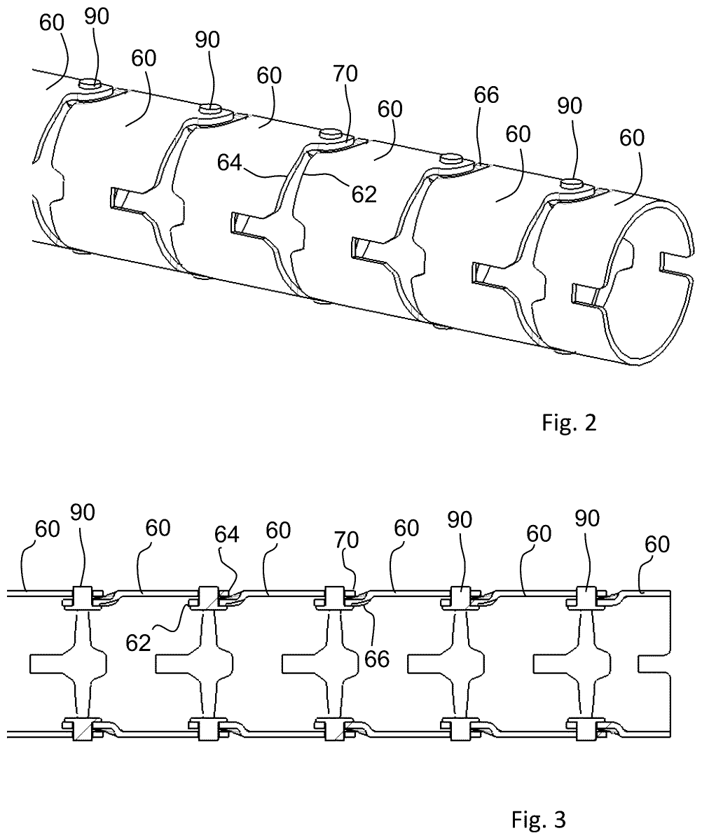

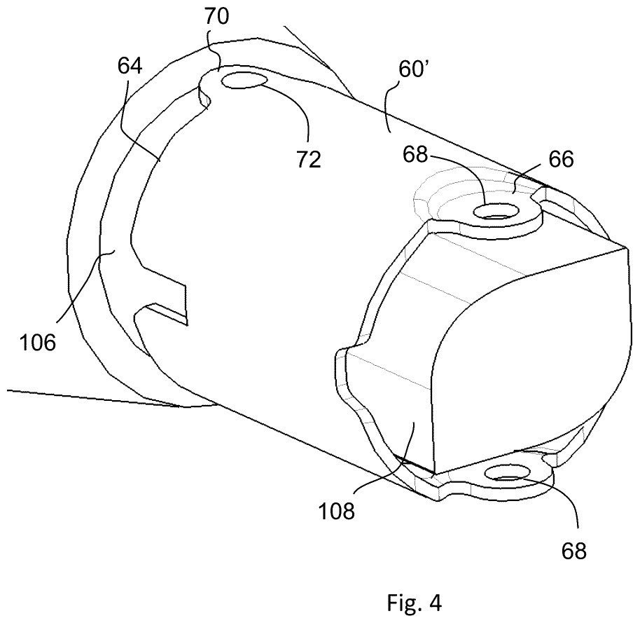

The articulation sections 60 are disposed adjacent to each other and are connected by rivets 90 so as to provide an articulated connection (FIGS. 2 and 3). The articulation sections 60 may have an identical configuration or this configuration may vary based on different size or other considerations. In the example of the method according to this disclosure essentially identical articulation section 60 are connected to each other based on principles of the method according to this disclosure.

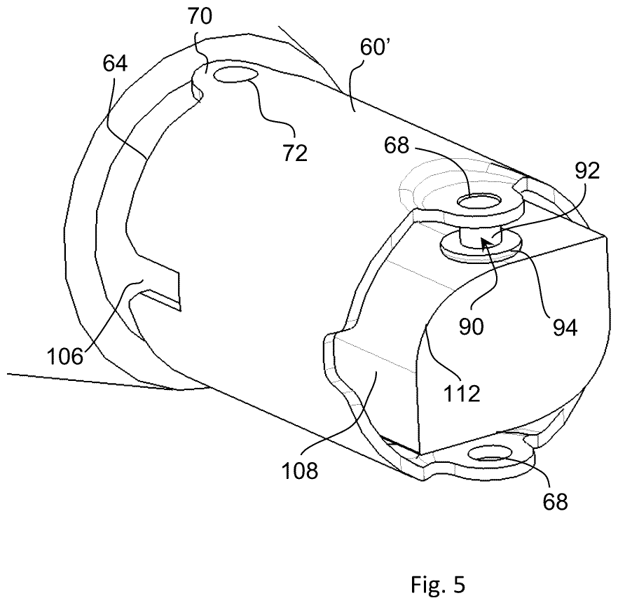

As can be seen in FIG. 4 a next to be connected articulation section (first articulation section) 60′, which is to be connected to a further adjacent articulation section (second articulation section) 60″, is placed on a mandrel portion 106 of the cam assembly tooling mandrel and loading pin 100. The cam assembly tooling mandrel and loading pin 100 has the mandrel portion 106, for supporting the cylindrical inner surface of the next to be connected articulation section 60′ and a loading pin portion 108, that has a cam surface 112 discussed further below. The first articulation section 60′ can rotate with the mandrel portion 106 and the loading pin portion 108 or the mandrel portion 106 and the loading pin portion 108 may rotate relative to the first articulation section 60′, in particular the first articulation section 60′ is held stationary. This next to be connected articulation section 60′ is disposed with the first side connection edge 62 having the inner ears 66 at upper and lower locations, exposed for connection. The cam assembly tooling mandrel and loading pin 100 is shown with the to be connected articulation section 60′ on the mandrel portion 106. A device as shown in FIG. 11 includes the cam assembly tooling mandrel and loading pin 100 that is supported by support block 120 with a support portion (widened portion) 124 and is rotatable by actuator 122 as indicated by rotation arrow 126.

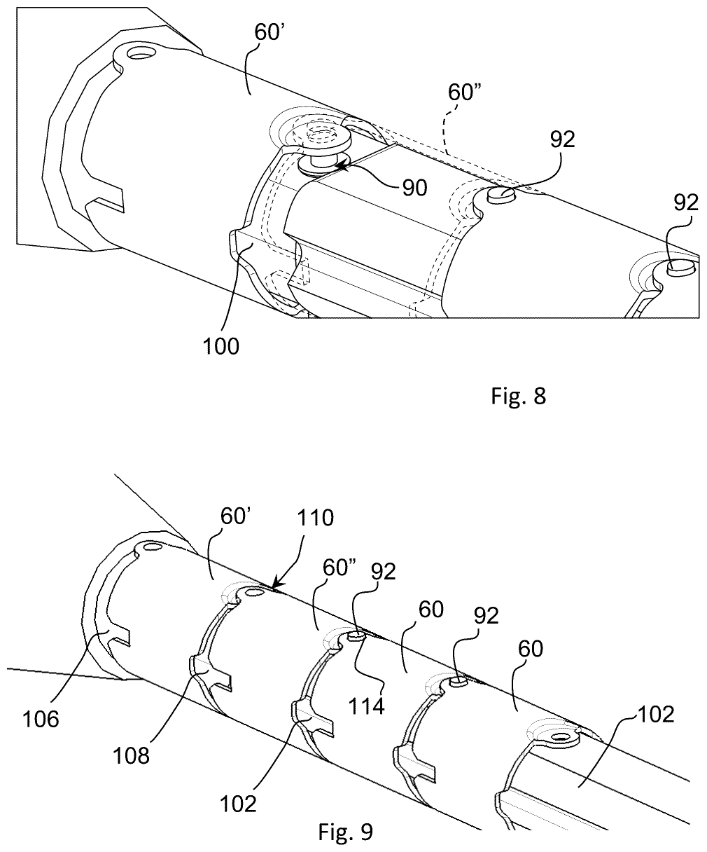

As can be seen in FIG. 5, a rivet 90 is positioned with a rivet shaft (cylindrical body) 92 in the inner rivet receiving hole 68 of the next to be connected articulation section 60′. The positioned rivet head 94 of the rivet 90 is shown adjacent to and in contact with the cam surface 112 of the loading pin 108. FIGS. 8, 9 and 10 show further articulation sections 60. An adjacent (left most) further articulation section 60″ has the second side connection edge 64 facing the first side connection edge 62 of next to be connected articulation section 60′. The second side connection edge 64 has the outer rivet receiving hole 72 of the outer ear 70.

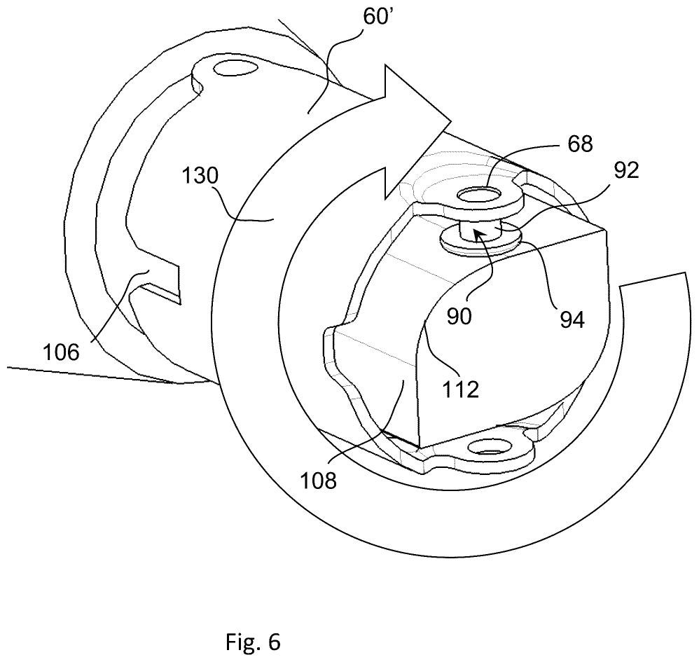

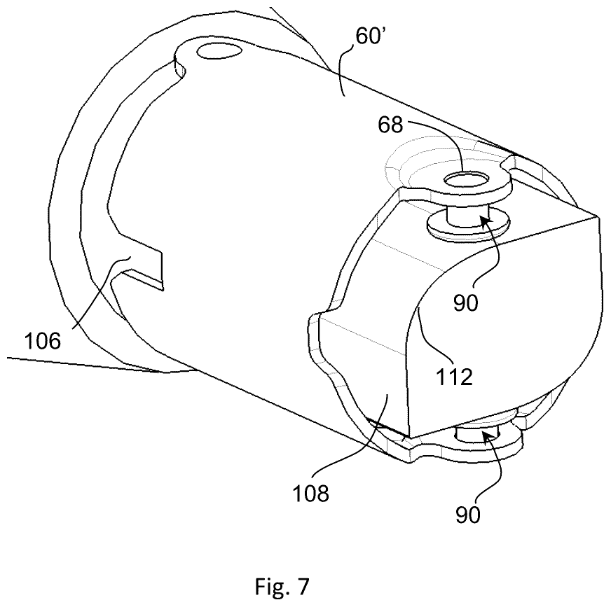

FIG. 6 shows the same state of the arrangement of articulation section 60′ to be connected on the cam assembly tooling mandrel and loading pin 100 and indicates a step of rotating the articulation section 60′ to be connected with the cam assembly tooling mandrel and loading pin 100 as shown by arrow 130. From the state shown in FIG. 6, the next to be connected articulation section 60′ is rotated with the cam assembly tooling mandrel and loading pin 100 by 180° to position the next to be connected articulation section 60′ in a position as shown in FIG. 7. A rivet shaft 92 of another rivet 90 is inserted in another inner rivet receiving hole 68, at the opposite side of the first side connection edge 62. After the other rivet 90 is positioned as shown in FIG. 7, two rivets are disposed in the respective inner rivet receiving holes 68 (one at top, one at bottom). Although the method may include rotation of articulation section 60 to be connected with the assembly tooling mandrel and loading pin 100, this is not required as the second rivet may be simply inserted at the lower rivet receiving hole 68 without rotation of the arrangement of articulation section 60′ to be connected with the cam assembly tooling mandrel and loading pin 100.

FIGS. 8 and 9 show the further step of placing the further articulation sections 60 at a location immediately adjacent to the next to be connected articulation section 60′. In FIG. 8, the left most further articulation section or adjacent further articulation section 60″ is shown with dashed line (as transparent). The further articulation sections 60 have already had rivets positioned and are shown with respective rivets in a pre-weld connected state 114. These further articulation sections 60 are carried by a support mandrel (second mandrel) 102 and positioned at the location with the left most (adjacent) further articulation section 60″ immediately adjacent to the next to be connected articulation section 60′. This positions the adjacent further articulation section 60″, carried on support mandrel 102, such that the outer rivet receiving hole 72 of the outer ear 70 of adjacent further articulation section 60″ is aligned with the inner rivet receiving hole 68 of the inner ear 66 of the next to be connected articulation section 60′. This provides a pre-assembly 110 with the articulation section 60′ to be connected supported by the mandrel portion 106 of the assembly tooling mandrel and loading pin 100 with the cam surface 112 facing and preferably contacting the two rivet heads 94, and with the adjacent (left most) further articulation section 60″, supported by support mandrel 102.

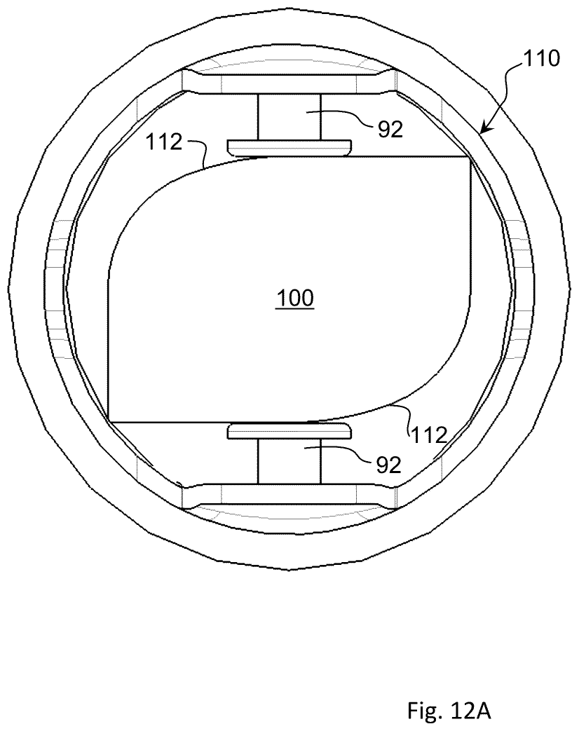

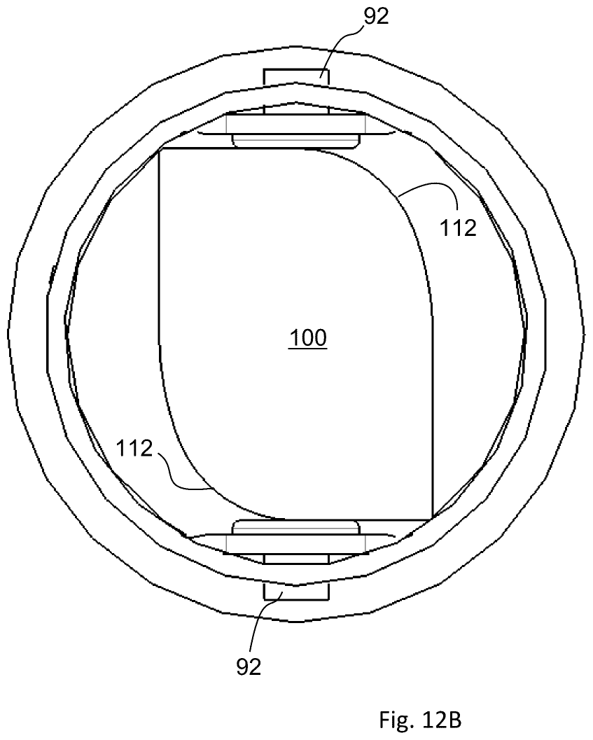

FIG. 11 shows the pre-assembly 110, including the next to be connected articulation section 60′ and adjacent further articulation section 60″ with aligned holes 68, 72 and with respective rivets 90 inserted only into the inner rivet receiving holes 68. This pre-assembly 110 is also shown in FIG. 12A in cross-section. As indicated in FIG. 11 the actuator 122 of support block 120 is rotated as indicated by rotation arrow 126. This rotates the assembly tooling mandrel and loading pin 100 such that the loading pin 108 with cam surface 112 rotates. The to be connected articulation section 60′ is held stationary, such that the loading pin portion 108 with cam surface 112 rotates relative to the to be connected articulation section 60′ and adjacent further articulation section 60″ with aligned holes 68, 72. The connected articulation section 60′ may be held stationary by an additional feature. The support mandrel may be configured to prevent rotation of the further articulation sections 60 including adjacent further articulation section 60″. The positioning of the adjacent further articulation section 60″, carried on support mandrel 102, such that the outer rivet receiving hole 72 of the outer ear 70 of adjacent further articulation section 60″ is aligned with the inner rivet receiving hole 68 of the inner ear 66 of the next to be connected articulation section 60′ has the effect of holding the next to be connected articulation section 60′ essentially in a stationary position (stationary). This allows for the rotation of the assembly tooling mandrel and loading pin 100 relative to the to be connected articulation section 60′.

FIG. 12B shows the loading pin 108 rotated, which results in the cam surface 112 pressing against the head 94 of the rivet 90 at each side. This rotation by 90° of the loading pin 108 results in each rivet shaft 92 being pressed through the inner rivet receiving hole 68 of the inner ear 66 and through the outer rivet receiving hole 72 of the outer ear 70. The cam surface 112 of the loaded pin 100 is pressed against the head 94 of each rivet 90 so as to position each rivet 90 in a pre-weld position at a known location with known clearance as shown in FIG. 12B.

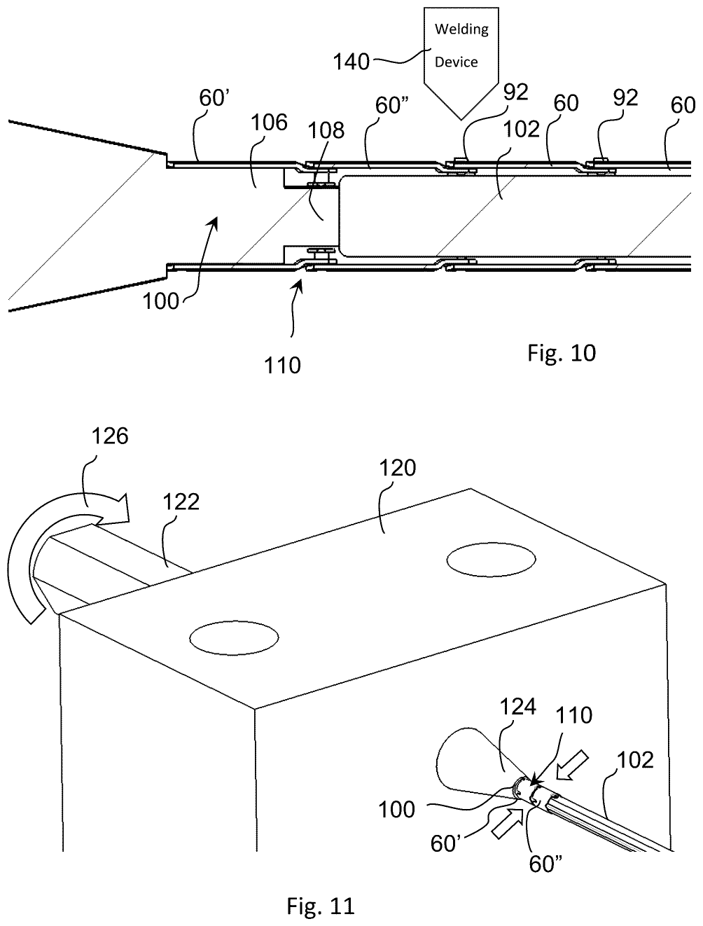

With each rivet 90 in its pre-weld position at the known location, each rivet 90 is fixedly connected to the adjacent further articulation section 60, in particular to the outer ear 70. Advantageously, the fixing of each rivet 90 to the adjacent articulation section comprises welding the rivet to the further adjacent articulation section 60″ at a region of the rivet shaft (cylindrical body) 92 adjacent to the outer ear 70 by a welding device 140. The next to be connected articulation section 60′ with each rivet 90 in a pre-weld position may be moved from the assembly tooling mandrel and loading pin having a cam surface 100 to the connected articulation sections support mandrel 102. Preferably, each articulated connection is brought to first a pre-weld connected state 114 and subsequently each rivet shaft 92 of each rivet 90 is welded about the peripheral edge of the respective rivet shaft 92 to an adjacent surface area of a respective outer ear 70. This may be done to all of the rivets at one side of the pre-weld chain of connected articulated sections 60. The connected articulated sections 60, in the pre-weld connected state 114, are welded, one after another, with the welding device 140 (FIG. 10) and subsequently the rivets at the other side of the chain of connected articulated sections, in the pre-weld connected state 114, are welded, one after another with the welding device 140. The connected articulated sections in the pre-weld connected state 114 may advantageously be supported by the second mandrel or support mandrel 102 as the welding device 140 is moved relative to the mandrel 102 to successively weld each rivet 90 to a respective outer ear 70 at one side. The support mandrel 102 is then rotated and the welding device 140 is moved relative to the mandrel 102 to successively weld each rivet 90 to a respective outer ear 70 at the opposite side.

The fixedly connected articulation sections 60 form a fixedly joined articulation section chain that comprises a plurality of such articulation sections 60, one after another. As noted, these articulation sections 60 may be of identical configuration but may also have varying axial extent. The first connection edges 62 of the articulation sections 60 may all be identical and the second connection edges 64 of the articulation sections 60 may be identical except for the distal last second side connection edge 64, which may have a different configuration (see FIGS. 2 and 3). Further, the first connection edge 62 of the chain of articulation section 60 may connect to a shaft section having a different configuration, namely without a second section connection edge 64 as this differently configured shaft section is not in the chain and acts to form the first articulated joint.

Although upper and lower rivets 90 are shown connecting each of the articulation sections 60 to be connected and adjacent further articulation section 60 after positioned with aligned holes 68 and 72, it may be possible to implement the flexible shaft at least partially with a single rivet at each articulation. However, the invention advantageously allows plural rivets to be accurately positioned simultaneously based on a single rotational movement of the cam assembly tooling mandrel and loading pin 100 relative to the first articulation section 60′. The disclosed method is particularly advantageous as to accurately positioning first and second rivets for welding without a need to linearly move the articulation sections 60 along with positioned rivets 90 along an assembly mandrel.

While specific embodiments of the invention have been shown and described in detail to illustrate the application of the principles of the invention, it will be understood that the invention may be embodied otherwise without departing from such principles.

### LIST OF REFERENCE NUMBERS

10 endoscope
12 handle/control
14 shaft
16 flexible/deflection section
20 control system 20
26 actuator
30 objective head
40 port
32 cover
50 electrical cable
60 articulation sections
60′ next to be connected articulation section (first articulation section)
60″ adjacent further articulation section (second articulation section)
62 first side connection edge
64 second side connection edges
66 inner ear (recessed rivet receiving ring portion)
68 inner rivet receiving hole
70 outer ear (outer rivet receiving ring portion)
72 outer rivet receiving hole
90 rivet (articulation hinge point)
92 rivet shaft (cylindrical body)
94 rivet head
100 cam assembly tooling mandrel and loading pin having a cam surface
102 connected articulation sections support mandrel
106 mandrel portion of the cam assembly tooling mandrel and loading pin
108 loading pin portion of the cam assembly tooling mandrel and loading pin
110 pre-assembly of positioned first articulation section and second articulation section
112 cam surface
114 pre-weld connected state
120 cam assembly tooling support block
122 actuator
124 cam assembly tooling mandrel widened portion
126 rotation of actuator to rotate loading pin relative to pre-assemble of positioned first articulation section and second articulation section
130 rotation of cam assembly tooling mandrel and loading pin and first articulation section assembly
140 welding device

What is claimed is:

1. A method comprising:
providing a first articulation section comprising a first section connection edge with an inner ear, comprising a recessed rivet receiving portion having an inner rivet receiving hole, and comprising an opposite section edge;
providing a second articulation section comprising a second section connection edge with an outer ear, comprising an outer rivet receiving portion having an outer rivet receiving hole, and comprising an opposite section edge;
placing the first articulation section on a cam assembly tooling mandrel and loading pin, the loading pin having a cam surface configured to contact with a head of a rivet;
inserting a rivet shaft of the rivet into the inner rivet receiving hole of the inner ear, wherein the head of the rivet is located at an interior side of the inner ear and facing the cam surface;
placing the second articulation section adjacent to the first articulation section with the outer rivet receiving hole of the outer ear aligned with the inner rivet receiving hole of the inner ear;
rotating the loading pin relative to the first articulation section to press the cam surface of the loading pin against the head of the rivet to move the rivet shaft partially through inner rivet receiving hole of the inner ear and into and partially through the outer rivet receiving hole of the outer ear; and
fixedly connecting the rivet to the second articulation section.

2. A method according to claim 1, wherein fixedly connecting the rivet to the second articulation section comprises welding the rivet to the outer ear of the second articulation section.

3. A method according to claim 1, wherein:
the first section connection edge of the first articulation section further comprises another inner ear, comprising another recessed rivet receiving portion having an another inner rivet receiving hole;
the second section connection edge of the second articulation section further comprises another outer ear, comprising an another outer rivet receiving portion having another outer rivet receiving hole; and
another rivet is provided.

4. A method according to claim 3, further comprising inserting another rivet shaft of the other rivet into the other inner rivet receiving hole of the other inner ear, wherein another head of the other rivet is located at an interior side of the other inner ear and facing the cam surface, wherein the cam surface is configured to contact with the head of the rivet and also to contact the head of the other rivet, whereby the rotation of the loading pin presses the cam surface of the loading pin against both the head of the rivet to move the rivet shaft into and partially through the outer rivet receiving hole of the outer ear and also the other head of the other rivet to move the other rivet shaft into and partially through the other outer rivet receiving hole of the other outer ear.

5. A method according to claim 1, further comprising the steps of providing a second mandrel configured to support the second articulation section to position and guide the second articulation section to a position adjacent to the first articulation section for said step of placing the second articulation section adjacent to the first articulation section with the outer rivet receiving hole of the outer ear aligned with the inner rivet receiving hole of the inner ear.

6. A method according to claim 5, wherein:
the second mandrel is configured to carry a plurality of further articulation sections;
the second articulation section is an adjacent further articulation section of the plurality of further articulation sections carried by the second mandrel; and
the plurality of further articulation sections carried by the second mandrel comprise connected articulation sections.

7. A method according to claim 6, wherein the connected articulation sections carried by the mandrel are connected in a pre-weld state.

8. A method according to claim 7, wherein:
fixedly connecting the rivet to the second articulation section comprises welding the rivet shaft to the outer ear of the second articulation section at a first side;
the welding of the rivet of the rivet shaft to the outer ear of the second articulation section at the first side is followed by welding each subsequent rivet shaft to the respective outer ear at the first side of the further articulation sections carried by the second mandrel; and
subsequent to welding rivet shafts to the outer ears of the further articulation sections at the first side, rivet shafts are welded to the outer ears of the further articulation sections at a second side.

9. A method according to claim 1, wherein:
the cam assembly tooling mandrel and loading pin comprises a mandrel portion that supports the first articulation section and a loading pin portion having the cam surface that is configured to contact with the head of the rivet;
the loading pin portion effects a pressing of the cam surface of the loading pin portion against the head of the rivet to move the rivet shaft partially through the inner rivet receiving hole of the inner ear and into and partially through the outer rivet receiving hole of the outer ear with the first articulation section held stationary relative to the cam assembly tooling mandrel and loading pin.

10. A method of forming fixedly connected articulation sections of an endoscope that has a shaft including a flexible portion comprised of the fixedly connected articulation sections, the method comprising:
providing a to be connected articulation section having a section distal or proximal edge with a first inner ear comprising a recessed rivet receiving portion having an inner rivet receiving hole and an second inner ear comprising a recessed rivet receiving portion having an inner rivet receiving hole and comprising a proximal or distal edge with a first outer ear comprising an outer rivet receiving portion having an outer rivet receiving hole and second outer ear comprising an outer rivet receiving portion having an outer rivet receiving hole;
providing a further articulation section adjacent to the to be connected articulation section, the further articulation section having a distal or proximal edge with a first inner ear comprising a recessed rivet receiving portion having an inner rivet receiving hole and a second inner ear comprising a recessed rivet receiving portion having an inner rivet receiving hole and comprising a proximal or distal edge with a first outer ear comprising an outer rivet receiving portion having an outer rivet receiving hole and a second outer ear comprising an outer rivet receiving portion having an outer rivet receiving hole;
placing the to be connected articulation section on a cam assembly tooling mandrel and loading pin, the loading pin having a cam surface configured to contact with a head of a first rivet and a head of a second rivet;
inserting a first rivet shaft of the first rivet into the inner rivet receiving hole of the first inner ear, wherein the first head of the first rivet is located at an interior side of the first inner ear and facing the cam surface;
inserting a second rivet shaft of the second rivet into the inner rivet receiving hole of the second inner ear, wherein the second head of the second rivet is located at an interior side of the second inner ear and facing the cam surface;
placing the further articulation section adjacent to the to be connected articulation section with the inner rivet receiving hole of the first inner ear aligned with the outer rivet receiving hole of the first outer ear and with the inner rivet receiving hole of the second inner ear aligned with the outer rivet receiving hole of the second outer ear;
rotating the loading pin relative to the to be connected articulation section to press the cam surface of the loading pin against the first head of the first rivet to move the first rivet shaft into and partially through the outer rivet receiving hole of the first outer ear and to simultaneously press the cam surface of the loading pin against the second head of the second rivet to move the second rivet shaft into and partially through the outer rivet receiving hole of the second outer ear;
fixedly connecting the first rivet to the further articulation section; and
fixedly connecting the second rivet to the further articulation section.

11. A method according to claim 10, wherein:
fixedly connecting the first rivet to the further articulation section comprises welding the first rivet to the first outer ear of the further articulation section; and
fixedly connecting the second rivet to the second articulation section comprises welding the second rivet to the second outer ear of the further articulation section.

12. A device for forming a chain of fixedly connected articulation sections of an endoscope with a shaft including a flexible portion comprised of the fixedly connected articulation sections, the device comprising:
a cam assembly tooling mandrel and loading pin, wherein the loading pin comprises a cam surface;
a cam assembly tooling support block supporting the cam assembly tooling mandrel and loading pin;
an actuator configured to rotate the loading pin relative to a pre-assembly of a positioned to be connected articulation section and a positioned further articulation section,
wherein the cam assembly tooling mandrel and loading pin is configured to support the to be connected articulation section comprising a first section connection edge with an inner ear comprising a recessed rivet receiving portion having an inner rivet receiving hole and comprising an opposite section edge,
wherein the further articulation section comprises a second section connection edge with an outer ear comprising an outer rivet receiving portion having an outer rivet receiving hole, and comprises an opposite section edge that is positionable adjacent to the to be connected articulation section with the outer rivet receiving hole of the outer ear aligned with the inner rivet receiving hole of the inner ear,
wherein the cam surface is configured to contact a head of a rivet having a rivet shaft of the rivet that is insertable into the inner rivet receiving hole of the inner ear, wherein the head of the rivet is located at an interior side of the inner ear and facing the cam surface;
wherein with the to be connected articulation section supported on the cam assembly tooling mandrel and loading pin, the loading pin is rotatable by the actuator to press the cam surface of the loading pin against the head of the rivet to move the rivet shaft partially through the inner rivet receiving hole of the inner ear and into and partially through the outer rivet receiving hole of the outer ear; and
means for fixedly connecting the rivet to the further articulation section.

13. A device according to claim 12, wherein the cam assembly tooling mandrel and loading pin comprises a mandrel portion configured to support the to be connected articulation section and a loading pin portion comprising the cam surface.

14. A device according to claim 12, further comprising a second mandrel configured to support the further articulation section to position and guide the further articulation section to position the further articulation section adjacent to the to be connected articulation section with the outer rivet receiving hole of the outer ear aligned with the inner rivet receiving hole of the inner ear.

15. A device according to claim 14, wherein:
the second mandrel is configured to carry a plurality of further articulation sections;
the further articulation section is an adjacent further articulation section of the plurality of further articulation sections carried by the second mandrel; and
the plurality of further articulation sections carried by the second mandrel comprise connected articulation sections.

16. A device according to claim 15, wherein the connected articulation sections carried by the mandrel are connected in a pre-weld connected state.

17. A device according to claim 16, further comprising a welding device configured to fixedly connect the rivet to the further articulation section, by welding the rivet shaft to the outer ear of the further articulation section at a first side and welding each subsequent rivet shaft to the respective outer ear at the first side of the further articulation sections carried by the second mandrel and to subsequently to weld rivet shafts to the outer ears of the further articulation sections at a second side.
